(19) United States
(12) Patent Application Publication
(10) Pub. No.: US 2003/0096850 A1
(43) Pub. Date: May 22, 2003

(54) Treating infections by administration of oxazolidinones

(76) Inventors: Charles W. Ford, Portage, MI (US); Jeffrey L. Watts, Kalamazoo, MI (US)

Correspondence Address: PHARMACIA & UPJOHN, 301 HENRIETTA ST, 0228-32-LAW, KALAMAZOO, MI, 49007, US

(21) Appl. No.: 10/266,304

(22) Filed: Oct. 8, 2002

Related U.S. Application Data
(60) Provisional application No. 60/328,665, filed on Oct. 11, 2001.

(51) Int. Cl. A61K 9/06; A61K 31/42; A01N 43/76

(52) U.S. Cl. 514/376; 424/400; 424/449

(57) ABSTRACT

Disclosed is a method of treating ear infections, soft-tissue infections, acne, or cellulitis in a mammal in need thereof comprising administration of Oxazolidinone in a pharmaceutical formulation or composition to the skin of the mammal at a site proximal to the site of the infection to deliver a pharmaceutically-effective amount of Oxazolidinone to the infection to have a concentration of the Oxazolidinone at the site of infection of about 0.5 to about 4 μg/ml, provided that the application for administration is not directly to the site of the infection.

### CROSS REFERENCE

This application claims the benefit of U.S. provisional application Serial No. 60/328,665 filed on Oct. 11, 2001, under 35 USC 119(e)(i), which is incorporated herein by reference in its entirety.

### BACKGROUND OF THE INVENTION

1. Field of the Invention

The present invention is a method of treating gram-positive infections by applying an Oxazolidinone to the skin of a mammal in need thereof to obtain a concentration of about 0.5 to about 4 μg/ml of the Oxazolidinone for about 40 to about 60% of the duration of dosage interval at the site of infection that is a site other than the site of application. Said method is particularly useful for treating infections including any one or more of the following: ear infections, soft-tissue infections, acne, or cellulites.

2. Background

The following US patents disclose various oxazolidinone antibiotics which are well known to those skilled in the art: U.S. Pat. Nos. 5,698,574; 5,688,792; 5,652,238; 5,627,181; 5,565,571; 5,231,188; and 5,164,510.

U.S. Pat. No. 6,040,306 discloses a method of treating psoriasis, arthritis and reducing the toxicity of cancer chemotherapy.

U.S. Pat. No. 5,688,792 discloses various oxazolidinone antibiotics effective against a number of human and veterinary pathogens.

WO 99/62504 discloses topical administration of oxazolidinones for transdermal delivery.

WO 97/42954 discloses a method of topically administering antimicrobial agents such as premafloxacin, premafloxacin-like compound, premafloxacin ester, ciprofloxacin, enrofloxacin, cefquinome, gentamicin or erythromycin for the treatment of systemic bacterial diseases in mammals.

WO 90/00407 A1 discloses oxazolidinone penetration enhancing compounds.

WO 85/00108 discloses a method of treating Acne Vulgaris which comprises the topical application of a preparation comprising an antibacterial agent in DMSO. The antibacterial agents include ara-A, acylovir, ribavirin, amikacin, cefamandole, cefoxitin, erythromycin, tetracycline, tobramycin, lincomycin and carbenicillin.

U.S. Pat. No. 3,743,727 discloses a method of enhancing tissue penetration of antimicrobial agents selected from the group consisting of sulfonamides, penicillins, antiviral agents and antibiotics produced by species of streptomyces by conjointly applying the agent to the tissue with DMSO.

U.S. Pat. No. 4,943,435 discloses a transdermal patch for delivering nicotine for prolonged periods of 12-24 hours.

### SUMMARY OF INVENTION

An aspect of the present invention is a method of treating infections that are other than at the surface of the skin including ear infections, soft-tissue infections, acne, or cellulitis in a mammal in need thereof which comprises administration of a pharmaceutical formulation containing a pharmaceutically-effective amount of an Oxazolidinone that penetrates across the skin barrier to obtain a concentration of about 0.5 to about 4 μg/ml of the Oxazolidinone for about 40 to about 60% of the duration of dosage interval at the site of infection that is a site other than the site of application.

Another aspect of the present invention includes the administration of an Oxazolidinone in combination with other antibiotic agents including, but not limited to, cephalosporin, aminoglycoside, or penem any of which of these other antibiotic agents may be administered orally, parentarally, or intravenously. It is preferred that these other antibiotic agents are administered to deliver 1-10 mg/kg/day for an adult. One of ordinary skill in the art will know the dosage and route of administration for these other antibiotic agents.

Another aspect of the present invention includes the method of treating ear infections, soft-tissue infections, or acne in a mammal in need thereof comprising administration of Oxazolidinone in a pharmaceutical formulation or composition to the skin of the mammal at a site proximal to the site of the infection to deliver a pharmaceutically-effective amount of Oxazolidinone to the infection to reach a concentration of the Oxazolidinone at the site of infection of about 0.5 to about 4 mg/ml.

Another aspect of the present invention includes the use of Oxazolidinone to prepare a medicament in a pharmaceutical formulation or composition for treating ear infections, soft-tissue infections, or acne in a mammal in need thereof comprising administration of the pharmaceutical formulation or composition to the skin of the mammal at a site proximal to the site of the infection to deliver a pharmaceutically-effective amount of Oxazolidinone to the infection to reach a concentration of the Oxazolidinone at the site of infection of about 0.5 to about 4 mg/ml.

The Oxazolidinones of the present invention are known: Examples 1-3. It is preferred that the Oxazolidinone be selected from:

(S)-N-[[3-[3-fluoro-4-[4-(hydroxyacetyl)-1-piperazinyl]-phenyl]-2-oxo-5-oxazolidinyl]methyl]acetamide,

(S)-N-[[3-[3-fluoro-4-(4-morpholinyl)phenyl]-2-oxo-5-oxazolidinyl]methyl]acetamide, or

[4(S)-cis]-(−)-N-[[3 -[3 -fluoro-4-(tetrahydro-1-oxido-2H-thiopyran-4-yl)phenyl]-2-oxo-5-oxazolidinyl]methyl]acetamide.

It is preferred that the Oxazolidinone is (S)-N-[[3-[3-fluoro-4-(4-morpholinyl)phenyl]-2-oxo-5-oxazolidinyl]methyl]acetamide.

The Oxazolidinones of the present invention do not include macrocyclic immunomodulators that are made into an oxazolidinone pro-drug for delivery/administration.

The pharmaceutical formulation contains a pharmaceutically-effective amount of an Oxazolidinone that penetrates the skin to obtain a concentration of about 0.5 to about 4 μg/ml of the Oxazolidinone for about 40 to about 60% of the duration of dosage interval at the site of infection. Said pharmaceutical formulation does not have to include what are commonly known as penetration enhancers, e.g., dodecyl pyrrolidone. The pharmaceutical formulation of the present invention can include an Oxazolidinone of the present invention and inert material for making the cream, gel, ointment, etc. Alternatively, the pharmaceutical formulation of the present invention can include an Oxazolidinone of the present invention, inert material for making the cream, gel, ointment, etc., and an inert penetration enhancer. The Oxazolidinone of the present invention is separate and distinct from the penetration enhancer; they are two separate materials. The Oxazolidinone of the present invention is the active ingredient used to treat the mammal in need thereof. It is preferred that a penetration enhancer not be used. One of ordinary skill in the art is familiar with pharmaceutically-acceptable penetration enhancers and inert material for making pharmaceutical formulations, including creams, gels, and ointments.

### DETAILED DESCRIPTION OF THE INVENTION

Antimicrobial agents may be administered systemically by enteral (oral, rectal) or parenteral (intravenous, intramuscular, intrathecal) routes, or applied locally to a specific site of infection. Avery's Drug Treatment, 4th ed. (1997), p 1471 (hereinafter “Avery's”). Therefore, antibacterial agents have generally been administered enteral or parenteral to treat infections occurring at a site other than the site of application, e.g., systemic infections, or an infections below the surface of the skin. One reason is due to the low permeability of the antibiotic agents. Unfortunately, adverse side effects occur when antibiotics are administered enteral or parenteral, including nausea, and furthermore, doses must be higher than the therapeutically-effective amount to obtain systemic levels in the circulatory system taking into consideration metabolism of the administered agent. Another issue with enteral or parenteral administration is that the administered antibiotic must be transported to the site of infection; therefore, the mammal in need of treatment must have circulation sufficient to circulate the antibiotic throughout the body.

Antibiotics applied locally are applied directly to the site of the infection. Application of an antibacterial agent at the site of the infection is often referred to as local administration. “Local application generally achieves high concentrations at the site of infection with minimal systemic absorption.” Avery's, p 1475. Antibacterial agents applied locally are generally too toxic for systemic use and are used to treat bacterial infections by local application directly to the site of infection. Id. Generally, local or topical application of antibiotic agents is limited to treat bacterial infections commonly known to those skilled in the art as superficial infections, e.g., a cut or skinned knee. Superficial infections involve bacterial growth that does not require enteral or parenteral administration of antibacterial agents to eradicate it from the infected area. Superficial infections can be treated by local or topical administration, which is known to those skilled in the art as application of the antibacterial agent to the specific site of infection. Irrigation is similar to local application in that a wound is flushed with an antibacterial solution or suspension. With irrigation, “[s]uppression of growth often results but cure of the infection is generally not achieved. There is usually inadequate penetration into the interstices and recesses of the infected areas. Also, ease of access for irrigation implies ready access of exogenous organisms; for example, colonization by Pseudomonas aeruginosa of a sinus tract leading to a chronic osteomyelitis caused by S. aureus.” [Infectious Diseases, A Treatise of Infectious Processes, 5th Ed., p 229 (1994), hereinafter “ID Treatise”.]

Therefore, there is a need to treat serious (non-superficial) infections in tissue that is not readily accessible for local application of antibacterial agents.

Surprisingly, the Oxazolidinones of the present invention are very permeable and can cross the barrier of the skin to obtain a concentration of about 0.5 to about 4 μg/ml of the Oxazolidinone for about 40 to about 60% of the duration of dosage interval at the site of infection, where the site of infection is a site other than the site of application. The pharmaceutical formulations or compositions comprising an Oxazolidinones of the present invention do not necessarily require a penetration enhancer to cross the surface of the skin to treat an infection that is at a site other than the site of application.

With the Oxazolidinones of the present invention, it is understood that the application is to the surface of the skin but the bacterial infection (the infection to be treated) is not at the location on the skin where the Oxazolidinone is applied. The Oxazolidinone may have antibacterial activity at the site of application but that is incidental to the infection to be treated. The infection being treated within the scope of the present invention is not on the surface of the skin at the site of application. It is preferred that the site of application is proximal to the site of the infection but not at the site of infection. An example of a proximal site of application includes the following: there is a raised bump on the left hand of a human with the skin being unbroken and the Oxazolidinone is applied on the left hand on the area of the swelling. It is more preferred that the size of the site of application is about four to eight centimeters larger in diameter than the site of the infection. For example, if the infection manifests itself by swelling to a one-centimeter diameter circle, application would occur on the surface of the skin above the swelling and the diameter of the application would be about five to nine centimeters.

Mammals within the scope of the present invention include humans, pets such as dogs and cats, or commercially important mammals such as horses, cattle, and pigs. It is preferred that the mammal be a human, dog or cat; more preferably a human.

The Oxazolidinones of the present invention are known: Examples 1-3. It is preferred that the Oxazolidinone be selected from:

(s)-N-[[3-[3-fluoro-4-[4-(hydroxyacetyl)-1-piperazinyl]-phenyl]-2-oxo-5-oxazolidinyl]methyl]acetamide,

(S)-N-[[3-[3-fluoro-4-(4-morpholinyl)phenyl]-2-oxo-5-oxazolidinyl]methyl]acetamide, or

[4(S)-cis]-(−)-N-[[3-[3-fluoro-4-(tetrahydro-1-oxido-2H-thiopyran-4-yl)phenyl]-2-oxo-5-oxazolidinyl]methyl]acetamide.

It is preferred that the Oxazolidinone be

(S)-N-[[3-[3-fluoro-4-(4-morpholinyl)phenyl]-2-oxo-5-oxazolidinyl]methyl]acetamide.

The infections treated by the present invention include ear infections, soft-tissue infections, acne, or cellulites. It is preferred that the amount of Oxazolidinone reaching the infected area is about 0.5 to about 4 μg/ml for about 40 to about 60% of the duration of dosage interval. The dosage interval is the amount of time between the sequential administrations of applications. Therefore, if the dosing is twice daily, or an application about every 12 hours, the Oxazolidinone should have a concentration of about 0.5 to about 4 μg/ml in the infected tissue for a minimum time of 4.8 hours or 40% of 12 hours at the intended site of treatment. A single administration is also within the scope of the present invention, as discussed herein. Under such circumstances, it is preferred that about thirty minutes be the dosage interval to allow penetration of the Oxazolidinone into the infected area that is separate and distinct from the site of application.

One type of infection to be treated within the scope of the present invention is an ear infection. Ear infections can be caused by either gram-positive or gram-negative bacteria or a mixture of both. A common ear infection is otitis media. Otitis media is an ear infection caused by inflammation of the middle ear. Avery's, p 565. The middle ear is composed of the tympanic cavity. The middle ear does not include the tympanic membrane, also known as the eardrum. Otitis media is distinguishable from otitis external, the latter of which involves infections of the external auditory canal and is generally treated with medications for different forms of dermatitis. Avery's, p 563. The two infections are distinctly different due to the location of the infected tissue and how infections of these tissue are treated. Compare pp 669-671 (discussing external otitis) with pp 671-678 (discussing otitis media). The scope of the present invention includes ear infections that are otitis media.

About 60% of the time, otitis media is caused by gram-positive bacteria, and in those cases the method of the present invention will be useful in treating the ear infection. In the remaining ear infections that occur about 40% of the time, using a combination treatment of Oxazolidinone with another agent will be useful in treating the ear infection. If not treated successfully, the two important consequences of ear infections are the ear drum can rupture leading to a reduction in hearing, and the surrounding tissues, including bone, can become infected and lead to a more life-threatening condition. Ear infections are most often caused by Streptococcus pneumoniae and sometimes by Streptococcus pneumoniae and Haemophilus influenzae at the same time. It is apparent to one skilled in the art that a mammal is in need of treatment for an ear infection when there is a fever and the mammal has pain in the ear or has pain in the ear and an ear exam discloses a swollen ear drum and fluid is observed behind the ear drum.

Ear infections, including otitis media, are treated by administering the desired Oxazolidinone directly to the skin proximal to the otitis media, e.g., to any one or more of the following: the eardrum, the external acoustic meatus, or the targus by use of a pharmaceutical formulation which includes a solution, suspension or emulsion. The present invention includes pharmaceutically-acceptable formulations to administer the Oxazolidinone for treatment of ear infections from about 0.1 to about 10% and also from about 0.2 to about 2%. It is preferred that the amount of Oxazolidinone reaching the infected tissue is about 0.5 to about 4 μg/ml for about 40 to about 60% of the duration of dosage interval. The Oxazolidinone is administered 2-4 times, preferably twice, daily for 314 days.

Soft-tissue infections are infections which are most often caused by staphylococci and streptococci. Such infections are very difficult to treat with known antibiotics because of their location and because treatment failures occur often requiring additional courses of therapy. Soft tissue “describes the extra skeletal connective tissue that accounts for more than 50 percent of body weight and includes muscle, tendon, fat, fascia, and synovium.”Oxford Textbook of Surgery, Morris, Peter J. and Malt, Ronald A., eds, (1994), p 1495. Fascia is defined as “as sheet or band of fibrous tissue such as lies deep to the skin or forms an investment for muscles and various organs of the body.” Dorland's Illustrated Medical Dictionary, 26th ed., p 487, hereinafter “Dorland's.” There are many types of fasciae. See, e.g., Dorland's, pp 487-488. Subcutaneous means “beneath the skin.” Dorland's, p 1263 (emphasis added). Synovia is “a transparent alkaline viscid fluid, resembling the white of an egg, secreted by the synovial membrane, and contained in joint cavities, bursae, and tendon sheaths.” Dorland's, p 1301. Bursae is “a sac or sac-like cavity filled with a viscid fluid and situated at places in the tissues at which friction would otherwise occur.”Dorland's, p 198.

It is apparent to one skilled in the art that a subject is in need of treatment for a soft-tissue infection when the subject has an inflamed, reddened, or tender area of the body under broken or unbroken skin and which is coupled with a fever. Soft-tissue infections are distinguishable from superficial infections because of the presence of a fever with soft-tissue infections and the lack of a fever with superficial infections. The present invention does not include treatment of superficial infections. Soft-tissue infections are treated by administering the desired Oxazolidinone to the skin proximal to the infected area by use of the appropriate pharmaceutical dosage form. It is preferred that the Oxazolidinone be administered in the form of a pharmaceutical formulation including a cream, ointment, gel, emulsion, suspension, solution, or patch, and it is preferred that the pharmaceutical formulation be administered 2-4 times daily, preferably 3 times daily, until 24 hours after the body temperature returns to normal and the redness, swelling and/or inflammation are(is) gone. The present invention includes pharmaceutically-acceptable formulations to administer the Oxazolidinone for treatment of ear infections from about 0.2 to about 40% and also from about 0.4 to about 10%. It is preferred that the amount reaching the infected tissue is about 0.5 to about 4 μg/ml for about 40 to about 60% of the duration of dosage interval. Other sanitary precautions should be utilized as are known to those skilled in the art.

A specific example of these soft-tissue infections is diabetic foot. Diabetic foot is an infection in the foot of a diabetic whose foot receives minor trauma in the presence of peripheral neuropathy and/or vascular disease causing the formation of an ulcer. Avery's, p 742. These same patients can have spreading cellulitis or more deep-seated infection to require parenteral antibiotic therapy. Avery's, p 742. Cellulitis is defined as “diffuse inflammation of the soft or connective tissue due to infection, in which a thin, watery exudates spreads through the cleavage places of interstitial and tissue spaces; it may lead to ulceration and abscess.” Dorland's, p 241. Diabetic polyneuropathy is sensory denervation and prevents those with diabetes from sensing trauma from causes including ill-fitting shoes or pebbles within shoes. Merck Manual of Diagnosis and Therapy, Century Edition, p 169, 1999 [hereinafter “Merck Manual”]. This lack of sensory perception results in the lack of detection of injuries which become infected, which left untreated become ulcerated and can necessitate amputation. Internal redness and swelling are the initial signs of infection before open wounds occur in the foot of a diabetic. This initial infection is below the surface of the skin has been treated with enteral or parenteral administration, which is complicated due the circulatory problems of mammals with diabetes. Therefore, there is a need for an administration near the site of infection.

For soft-tissue infections of the present invention, the Oxazolidinone is administered by use of a pharmaceutical formulation which is a cream, ointment, gel, emulsion, suspension, solution, or patch, preferably 3 times daily. The Oxazolidinone is applied to an area of the foot that is about one to three inches in circumference of the swollen/reddened area. The Oxazolidinone can also be applied to the entire area of the foot and to areas of the ankle and leg depending on the extent of the infection. The foot may have open ulcers if the infection is not treated at initial inception of the infection. The scope of the present invention is not intended to treat the open ulcers but to treat the infection of the soft tissues under the surface of the skin and open ulcers. The Oxazolidinone would have unintended beneficial effect on the ulcers, but the ulcers are not the intended site of infection to be treated with the present invention. The infections of the present invention are below the surface of the skin and under the ulcers; such infections cannot be treated with the agents currently applied locally.

Moreover, with proper monitoring of the diabetic mammal, the infections of diabetic foot can be detected before ulcerations occur at the surface of the foot. Once properly identified as a bacterial infection within the tissue within the foot and not on the surface of the skin of the foot, the infection can be treated as described herein.

The present invention to treat diabetic foot includes pharmaceutically-acceptable formulations to administer the Oxazolidinone for treatment of diabetic foot from about 0.1 to about 10% and also from about 0.2 to about 6%. It is preferred that the amount of Oxazolidinone reaching the infected tissue is about 0.5 to about 4 μg/ml for about 40 to about 60% of the duration of dosage interval. It is preferred that the Oxazolidinone be applied 2-4 times daily. It is more preferred that the Oxazolidinone be applied 3 times daily.

When the soft-tissue infection is diabetic foot, the Oxazolidinone can be administered in combination with other antibiotic agents including, but not limited to, cephalosporin, aminoglycoside, or penem. These other antibiotic agents are administered orally, parentarally, intravenously. It is preferred that these other antibiotics are administered to deliver 1-10 mg/kg/day for an adult. Antibacterial agents currently used to treat diabetic foot are flucloxacillin, cefalexin, metronidazole, amoxicillin and clavulanic acid, clindamycin, ciprofloxacin, fusidic acid, and rifampicin. Avery's, p 742. Existing treatment of diabetic foot administers the antibacterial agent orally or intravenously. Merck Manual, pp 1103, 1106, 1110, 1113-1114, 1118, and 1120.

Soft-tissue infections of the present invention also include abscesses. An abscess is “a localized collection of pus in a cavity formed by the disintegration of tissues.”Dorland's, p. 4. An acute abscess is an abscess “which runs a relatively short course, producing some fever and a painful local inflammation.” Id. The abscess is below the surface of the skin. See Id., diagram.

Soft-tissue infections of the present invention also include erysipelas. Erysipelas is “a contagious disease of skin and subcutaneous tissue due to infection with Streptococcus pyogenes and marked by redness and swelling of affected areas, with constitutional symptoms; sometimes accompanied by vesicular and bullous lesions.” Dorland's, p 460.

Another soft-tissue infection of the present invention includes infections that are caused by bacteria which are normally present within the skin. Such bacteria exist within the skin of mammals without causing an active infection when the immunity system of said mammal is stable and intact. Such bacteria are commonly called “normally metabolically quiescent bacteria.” Surgery, however, due to its invasive nature, permits those organisms to spread within the surgical area. The surgical area includes the skin cut to allow access to the body and the organs and tissue cut, e.g., the sternum and heart in heart surgery, and immediately surrounding tissue, e.g., the muscle and connective tissue in heart surgery.

Normal pre-surgical procedure includes cleaning the surface of the skin of the mammal undergoing surgery. However, due to bacteria living below the surface of the skin, e.g., below the surface of the skin including the dermis and hypodermis, eradication of bacteria below the epidermis cannot occur. During the surgery, the normally metabolically quiescent bacteria are spread throughout the surgical area. Due to the presence of serum and destroyed tissues, the bacteria are exposed to massive amounts of nutrients, replicate, and spread throughout the surgical area causing an active infection. The bacteria can also cause disease at sites distal to the surgical area by spreading through the blood stream when not promptly and properly treated. Currently, there is no pre-treatment to eradicate these normally metabolically quiescent bacteria from the surgical area.

Eradication of normally metabolically quiescent bacteria in the layers of the skin before surgery is an unmet need. The Oxazolidinones of the present invention can be useful to eradicate the normally metabolically quiescent bacteria through pre-treatment of the incision area. The incision area includes the specific site of incision and an area large enough to include approximately two to four centimeters beyond each point of incision. The incision area is to be treated with an Oxazolidinone in a pharmaceutically-acceptable formulation. One of ordinary skill in surgery will know what size of incision will be used for the intended surgery, and, therefore, will know where to apply the Oxazolidinone of the present invention. The pre-treatment prevents the normally metabolically quiescent bacteria from causing serious, potentially life-threatening infections after surgery. Due to surgery being of unknown need, e.g., emergency surgeries to remove an appendix, the treatment involves the application of Oxazolidinone from about 1 to about 3 time(s) before surgery occurs and from about 30 minutes to about 24 hours before surgery occurs.

For pre-surgery treatment, the Oxazolidinone is administered by use of a pharmaceutical formulation including a cream, ointment, gel, emulsion, suspension, solution, or patch. The present invention includes pharmaceutically-acceptable formulations to administer the Oxazolidinone for treatment of ear infections from about 0.1 to about 10% and also from about 0.2 to about 2%. It is preferred that the amount reaching the area of the skin to be cut to allow surgery is about 0.5 to about 4 μg/ml for about 40 to about 60% of the time after administration and before surgery, but at least for about 30 minutes before surgery is to occur.

Another aspect of the present invention includes acne. Acne is “[a] common inflammatory disease of the pilosebaceous glands characterized by comedones, papules, pustules, inflamed nodules, superficial pus-filled cysts, and (in extreme cases) canalizing and deep inflamed, sometimes purulent sacs.” Merck Manual, p 811.

Sebaceous glands are small saccular structures [ ] lying in the dermis and present over the whole body except the thick hairless skin of the palms, soles and flexor surfaces; they secrete an oily substance, . . . over the skin surface and on to hairs.

Typically, sebaceous glands consist of a cluster of two to five (occasionally up to 20) secretory acini opening by a short common duct into the apical portion of a hair follicle and can be considered together with the follicle and arrector pili muscle part of an anatomical unit, the pilo-sebaceous complex . . . In some areas of thin skin lacking hair follicles, their ducts open instead directly on to the skin surface, e.g., on the lips and corners of the mouth, nipples, female mammary areolae (Montgomery's tubercles), glans penis, inner surface of the prepuce (glands of Tyson), glans clitoridis and labia minora; at margins of the eyelids, the large complex palpebral tarsal glands (Meibomian glands) are of this type . . . [Gray's Anatomy, 37th Ed., 1989, p 92, hereinafter “Gray's”.]

Comedo is “[a] plug of keratin and sebum within the dilated orifice of a hair follicle, frequently containing the bacteria . . . ; also called a blackhead. Dorland's, p 292. Papule is “a small circumscribed, superficial, solid elevation of the skin.” Dorland's, p 961. Pustule is “a visible collection of pus within or beneath the epidermis, often in a hair follicle or sweat gland.” Dorland's, p 1098. Nodules is “a small boss or node which is solid and can be detected by touch; see also nodulus.” Dorland's, p 897. Nodulus is “a nodule or small knot; used in anatomical nomenclature as a general term to designate a comparatively minute collection of tissue.” Dorland's, p 898. Cyst means combining form denoting a relationship to a sac, cyst, or bladder.” Dorland's, p 340. Sac is “a pouch; a baglike organ or structure.” Dorland's, p1165.

The sebaceous glands infected in acne would be those opening into the hair follicle that becomes blocked upon infection, and, would be in the dermis below the surface of the skin. See, e.g., Gray's, pp 71, 90-91.

Acne vulgaris is caused by the anaerobic bacterium Propionibacterium acnes which is found in blocked and inflamed oil glands or ducts in the skin of humans, particularly teenagers. These infections occur well below the surface of the skin and need to be treated at that location. While not life threatening, serious acne can cause both skin scars and emotional trauma. It is preferred that the acne be treated with a pharmaceutical formulation including a cream, ointment, gel, emulsion, suspension, solution or patch. The present invention includes pharmaceutically-acceptable formulations to administer the Oxazolidinone for treatment of ear infections from about 0.1 to about 10% and also from about 0.2 to about 6%. It is preferred that the amount reaching the infected tissue is about 0.5 to about 4 μg/ml for about 40 to about 60% of the duration of dosage interval. The acne is treated 2-4 times, preferably 2 times, daily until the acne is contained to the satisfaction of the patient and treating physician.

Cellulitis refers to “diffuse inflammation of the soft or connective tissue due to infection, in which a thin, watery exudate spreads through the cleavage planes of interstitial and tissue spaces; it may lead to ulceration and abscess.” Dorland's Illustrated Medical Dictionary, 26^{th} Ed. (1981, p 241). It is preferred that cellulites is treated with a pharmaceutical formulation including a cream, ointment, gel, emulsion, suspension, solution or patch to penetrate the full thickness of the infection. The present invention includes pharmaceutically-acceptable formulations to administer the Oxazolidinone for treatment of cellulitis from about 0.1 to about 10% and also from about 0.2 to about 6%. It is preferred that the amount reaching the infected tissue is 4 μg/ml for about 40 to about 60% of the duration of dosage interval. The cellulitis is treated 2-4 times, preferably 2 times, daily for a period of about at least 10 days, but at the physician's discretion.

The gram-positive microorganisms which cause the infections treated by the Oxazolidinones of the present invention include staphylococci, streptococci, or eterococci. It is preferred that the infection be caused by staphyloccoci. The important species of these genius are Staphyloccus aureus, Staphyloccus epidermidis and Staphyloccus hemolyticus. The Oxazolidinones of the present invention also treat gram-negative infections caused by anaerobes such as Bacteroides fragilis. The Oxazolidinone can be combined with other antibiotics to treat infections caused by a broader spectrum of gram-negative and/or grampositive microorganisms.

By “treating an infection” including ear infections, soft-tissue infections, and acne in a warm-blooded mammal who is in need of such treatment, means the mammal has an infection which is causing it a problem, including a fever, pain such as ear ache, abscess, or inflammation of a tissue. Treating infections means administering to the mammal Oxazolidinone such that the mammal obtains sufficient concentration of the Oxazolidinone in the affected area to either kill the existing microorganisms and/or to stop them from growing to a point where the body's natural defense mechanism can reduce or eradicate the unwanted microorganisms. “Treating” also includes preventing an infection, or preventing a minor infection from growing into a larger infection especially with acne or pre-treatment for surgery. Even though the patient may not observe blocked or inflamed oil glands or ducts, or not feel ill before surgery, the antimicrobial agents may still be present but are less metabolically active. Treating a teenager who has had acne to prevent future occurrences is included within the scope of “treating” as used in this patent. Also, using a topical application of Oxazolidinone as a pre-surgical pre-treatment to eradicate bacterial microbes is within the scope of “treating” as used in this patent.

In the method of the present invention, the Oxazolidinones can be used either individually or in combination with each other. Further, they can be used in combination with other antibacterial agents which are being administered by oral or intravenous administration. The Oxazolidinone can be combined with other antibiotic compounds including, but not limited to, cephalosporin, aminoglycoside, or penem. In addition, the Oxazolidinone can be used with non-antibacterial agents in treating the infections of this invention.

The exact dosage and frequency of administration depends on the particular Oxazolidinone used; the particular condition being treated; the severity of the condition being treated; the age, weight, and general physical condition of the particular patient; and other medication the particular patient may be taking as is well known to those skilled in the art and can be more accurately determined by the patient's response to the particular treatment administered. If the treatment is in combination with oral, parenteral, or intravenous administration, the blood level or concentration of the other medicament(s) in the patient's blood can also be measured.

The Oxazolidinone of the present invention can be applied in the form of pharmaceutical compositions or formulations, including cream, ointment, gel, emulsion, suspension, solution, or patch.

### Definitions and Conventions

The definitions and explanations below are for the terms as used throughout this entire document including both the specification and the claims.

### Definitions

All temperatures are in degrees Centigrade.

THF refers to tetrahydrofuran.

DMF refers to dimethylformamide.

Saline refers to an aqueous saturated sodium chloride solution.

Chromatography (column and flash chromatography) refers to purification/separation of compounds expressed as (support, eluent). It is understood that the appropriate fractions are pooled and concentrated to give the desired compound(s).

Ether refers to diethyl ether.

TLC refers to thin-layer chromatography.

When solvent pairs are used, the ratios of solvents used are volume/volume (v/v).

When the solubility of a solid in a solvent is used the ratio of the solid to the solvent is weight/volume (wt/v).

The pharmaceutical compositions or formulations are discussed in percentages to reflect the ratio of the weight of the active ingredient to the volume of the other ingredients of the pharmaceutical compositions or formulations, including the excipient or carrier.

Pharmaceutically acceptable refers to those properties and/or substances which are acceptable to the patient from a pharmacological/toxicological point of view and to the manufacturing pharmaceutical chemist from a physical/chemical point of view regarding composition, formulation, stability, patient acceptance and bioavailability.

Oxazolidinone refers to the compounds of EXAMPLES 1-3 of the present invention. Because the treatment of the present invention includes using Oxazolidinones in combination with each other, the use of the singular word “Oxazolidinone” refers to either one of the identified Oxazolidinones or any combination of the identified Oxazolidinones.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### (S)-N-[[3-[3-Fluoro-4-[4-(hydroxyacetyl)-1-piperazinyl]-phenyl]-2-oxo-5-oxazolidinyl]methyl]acetamide

(S)-N-[[3-[3-Fluoro-4-[4-(hydroxyacetyl)-1-piperazinyl]-phenyl]-2-oxo-5-oxazolidinyl]methyl]acetamide is known, see U.S. Pat. No. 5,652,238, EXAMPLE 1.

### Example 2

### (S)-N-[[3-[3-Fluoro-4-(4-morpholinyl)phenyl]-2-oxo-5-oxazolidinyl]methyl]acetamide

(S)-N-[[3-[3-Fluoro-4-(4-morpholinyl)phenyl]-2-oxo-5-oxazolidinyl]methyl]acetamide is known, see U.S. Pat. No. 5,688,792, EXAMPLE 5.

### Example 3

### [4(S)-cis]-(−)-N-[[3-[3-Fluoro-4-(tetrahydro-1-oxido-2H-thiopyran-4-yl)phenyl]-2-oxo-5-oxazolidinyl]methyl]acetamide

A mixture of (S)-(−)-N-[[3-[3-fluoro-4-(3,6-dihydro-2H-thiopyran-4-yl)phenyl]-2-oxo-5-oxazolidinyl]methyl]acetamide S-oxide (International Publication No. WO 97/09328, 4.50 g) and platinum oxide (697 mg) in methanol (164 ml) is shaken on the Parr apparatus under a hydrogen atmosphere at 40 psi for 18 hours. The catalyst is then removed by filtration through Celite, and the filtrate is concentrated under reduced pressure and the residue chromatographed on silica gel (230-400 mesh, 350 g), eluting with a gradient of methanol/methylene chloride (3/97-7/93). The appropriate fractions (those fractions with an R_{f}=0.44 by TLC; methanol/chloroform, 10/90) are pooled and concentrated to give the title compound, mp=203-204°.

### Example A

### A Human who has Acne is Treated with an Oxazolidinone Ointment

A 14 year old 70 kilo male who has acne as evidenced by reddened and swollen pustules located over his face, neck, chest and back is treated by the administration of a ointment containing 30 mg/ml of (S)-N-[[3-[3-fluoro-4-[4-(hydroxyacetyl)-1-piperazinyl]-phenyl]-2-oxo-5-oxazolidinyl]methyl]acetamide administered twice daily until the redness and swelling are gone. After administration of the OXAZOLIDINONE, the reddened and swollen pustules are greatly reduced.

### Example B

### A Human who has an Ear Infection is Treated with Oxazolidinone Solution

A seven year old 28 kilo child has otitis media as evidence by the presence of a bulging ear drum with observed fluid behind it and has had a fever of 1020 for two days. Ten drops of a solution containing 10 mg/ml of (S)-N-[[3-[3-fluoro-4-(4-morpholinyl)phenyl]-2-oxo-5-oxazolidinyl]methyl]acetamide is dropped into the ear twice daily for ten days with an eye dropper and is immediately followed by closing the outer ear canal with cotton. Before 10 days, the fever is down and the pain is gone.

### Example C

### A Human is Treated with an Oxazolidinone Solution Prior to Surgery

A 48 year old 75 kilo male is being prepared for surgery; the types of surgeries include open-heart surgery, joint replacement, back surgery, or any type of invasive surgery. A cream or ointment preparation of 10-30 mg/ml (S)-N-[[3-[3-fluoro-4-(4-morpholinyl)phenyl]-2-oxo-5-oxazolidinyl]methyl]acetamide is liberally spread by a nurse wearing gloves upon the intended site of the incision and an area of 8 inches around the margins of the intended incision site the evening prior to surgery and the first thing in the morning of the surgery. The area of application is kept free of clothing until the cream or ointment is absorbed. After administration of the Oxazolidinone, the skin commensalpathogens Staphylococcal aureus and Staphylococcal epidermidis are killed or are prevented from growing throughout the full thickness of the skin at and adjacent to the site of the intended incision.

### Example D

### Treatment of Diabetic Foot

A 64 year old 70 kilo female with a history of diabetes and infected foot ulcerations, also known as diabetic foot, is treated with a cream or ointment preparation of 10-30 mg/ml (S)-N-[[3-[3-fluoro-4-(4-morpholinyl)phenyl]-2-oxo-5-oxazolidinyl]methyl]acetamide. The cream or ointment is liberally spread over the exposed surface of the ulcerations and adjacent whole skin three times daily for 10-14 days. Following the course of Oxazolidinone treatment, tissue destruction in the ulcerated area has visibly ceased and tissue repair has begun as evidenced by lack of serous-fluid “weeping” and reduction in swelling.

### Example E

A rodent has been inoculated with a gram-positive infection. The rodent's hair is removed from the skin that is above said infection. An Oxazolidinone pharmaceutical composition or formulation is applied to the shaven skin up to 4 times daily. The infection is eradicated.

What is claimed:

1. A method of treating ear infections, soft-tissue infections, or acne in a mammal in need thereof comprising administration of Oxazolidinone in a pharmaceutical formulation or composition to the skin of the mammal at a site proximal to the site of the infection to deliver a pharmaceutically-effective amount of Oxazolidinone to the infection to reach a concentration of the Oxazolidinone at the site of infection of about 0.5 to about 4 μg/ml.

2. The method according to claim 1, where the mammal is a human.

3. The method according to claim 1, where the mammal is a dog or cat.

4. The method according to claim 1, where the infection is an ear infection.

5. The method according to claim 4, where the ear infection is otitis media.

6. The method according to claim 5, where the pharmaceutical formulation is a solution, suspension, or emulsion.

7. The method according to claim 6, where the pharmaceutical formulation has a pharmaceutically-effective amount of Oxazolidinone from about 0.1 to about 10%.

8. The method according to claim 7, where the pharmaceutical formulation has a pharmaceutically-effective amount of Oxazolidinone from about 0.2 to about 2%.

9. The method according to claim 4, where the infection is soft-tissue infections.

10. The method according to claim 9, where the infection is diabetic foot.

11. The method according to claim 10, where the pharmaceutical formulation is a cream, ointment, gel, emulsion, suspension, solution, or patch.

12. The method according to claim 11, where the pharmaceutical formulation has a pharmaceutically-effective amount of Oxazolidinone from about 0.1 to about 10%.

13. The method according to claim 12, where the pharmaceutically effective amount of Oxazolidinone from about 0.2 to about 6%.

14. The method according to claim 11, where the treatment is the administration of Oxazolidinone and the co-administration of another antibiotic is cephalosporin, aminoglycoside, or penem, provided that cephalosporin, aminoglycoside, or penem is administered orally, parentarally, or intravenously to administer 1-10 mg/kg per day for an adult.

15. The method according to claim 1, where the infection is cellulitis.

16. The method according to claim 15, where the pharmaceutical formulation includes a cream, ointment, gel, emulsion, suspension, solution, or patch.

17. The method according to claim 16, where the cream, ointment, gel, emulsion, suspension, solution, or patch has a pharmaceutically-effective amount of Oxazolidinone from about 0.1 to about 10%.

18. The method according to claim 17, where the pharmaceutical formulation has a pharmaceutically-effective amount of Oxazolidinone from about 0.2 to about 6%.

19. The method according to claim 1, where the treatment is prior to surgery to eradicate normally metabolically quiescent bacteria in the skin of the mammal undergoing surgery.

20. The method according to claim 19, where the pharmaceutical formulation is a cream, ointment, gel, emulsion, suspension, solution or patch.

21. The method according to claim 20, where the pharmaceutical formulation has a pharmaceutically-effective amount of Oxazolidinone from about 0.1 to about 10%.

22. The method according to claim 21, where the pharmaceutical formulation has a pharmaceutically-effective amount of Oxazolidinone from about 0.2 to about 6%.

23. The method according to claim 1, where the infection is soft-tissue infections manifested by inflammation, reddening, and/or a tender area below the surface of the skin.

24. The method according to claim 23, where the pharmaceutical formulation is a cream, ointment, gel, emulsion, suspension, solution, or patch.

25. The method according to claim 24, where the pharmaceutical formulation has a pharmaceutically-effective amount of Oxazolidinone from about 0.2 to about 40%.

26. The method according to claim 25, where the pharmaceutical formulation has a pharmaceutically-effective amount of Oxazolidinone from about 0.4 to about 10%.

27. The method according to claim 1, where the infection is acne vulgaris.

28. The method according to claim 27, where the pharmaceutical formulation is a cream, ointment, gel, emulsion, suspension, solution, or patch.

29. The method according to claim 28, where the cream, ointment, gel, emulsion, suspension, solution, or patch has a pharmaceutically-effective amount of Oxazolidinone from about 0.1 to about 10%.

30. The method according to claim 29, where the pharmaceutical formulation has a pharmaceutically-effective amount of Oxazolidinone from about 0.2 to about 6%.

31. The method according to claim 1, where the infection is caused by staphylococci, streptococci, or enterococci.

32. The method according to claim 31, where the infection is caused by staphylococci.

33. The method according to claim 1, where the Oxazolidinone is:
(S)-N-[[3-[3-fluoro-4-[4-(hydroxyacetyl)-1-piperazinyl]-phenyl]-2-oxo-5-oxazolidinyl]methyl]acetamide,
(S)-N-[[3-[3-fluoro-4-(4-morpholinyl)phenyl]-2-oxo-5-oxazolidinyl]methyl]acetamide, or
[4(S)-cis]-(−)-N-[[3-[3-fluoro-4-(tetrahydro-1-oxido-2H-thiopyran-4-yl)phenyl]-2-oxo-5-oxazolidinyl]methyl]acetamide.

34. The method according to claim 31, where the Oxazolidinone is (s)-N-[[3-[3-fluoro-4-(4-morpholinyl)phenyl]-2-oxo-5-oxazolidinyl]methyl]acetamide.
